(19) United States
(12) Patent Application Publication
(10) Pub. No.: US 2004/0022775 A1
(43) Pub. Date: Feb. 5, 2004

(54) Methods of treating viral infections in mammals

(76) Inventors: Gregor Reid, London (CA); Andrew W. Bruce, Toronto (CA); Yong Kang, Longon (CA)

Correspondence Address: SCULLY SCOTT MURPHY & PRESSER, PC, 400 GARDEN CITY PLAZA, GARDEN CITY, NY, 11530

(21) Appl. No.: 10/413,993

(22) Filed: Apr. 15, 2003

Related U.S. Application Data
(60) Provisional application No. 60/372,705, filed on Apr. 15, 2002.

(51) Int. Cl. A61K 45/00

(52) U.S. Cl. 424/93.45

(57) ABSTRACT

This invention relates to methods of preventing and treating viral infections or inhibiting the spread of viruses by administering compositions of at least one Lactobacillus whole cell or by-product thereof to patients in need of such treatment.

### CROSS REFERENCE TO RELATED APPLICATIONS

The present application claims benefit of U.S. Provisional Application No. 60/372,705 filed Apr. 15, 2002.

### FIELD OF THE INVENTION

This invention relates to methods of creating a healthy vaginal tract such that viruses, e.g. HIV, vesicular stomatitis virus, adenovirus, herpesvirus, papilloma viruses do not infect the host or spread from the host to the sexual partner, said method comprising the administration of compositions of at least one Lactobacillus organism or by-product thereof.

### BACKGROUND OF THE INVENTION

The depletion of vaginal lactobacilli has been associated with an increased risk of urogenital infections including bacterial vaginosis (BV) and acquisition of Human Immunodeficiency Virus (HIV)(Reid & Bruce 2001. J. Infect. Dis. 183(S1):S77-80). Therefore, restoration of the flora by vaginal instillation of probiotic lactobacilli could lead to a reduced incidence and/or spread of infections, including those caused by viruses such as HIV, vesicular stomatitis virus (VSV), papilloma viruses and human adenoviruses, among others. The prevention of viral transmission through lactobacilli production of hydrogen peroxide has been analyzed (Alvarez-Olmos & Oberhelman 2001. Clin. Infect. Dis. 32: 1567-76).

However, prior to the present invention, the art has not recognized that metabolic by-products from Lactobacillus strains, including strains such as Lactobacillus rhamnosus GR-1, which does not produce hydrogen peroxide, have an inhibitory effect on human cells which reduces the risk of shedding of viruses upon sexual contact. Furthermore, the prior art has failed to appreciate that Lactobacillus whole cells and metabolic by-products thereof can also kill DNA and RNA viruses within seconds of exposure.

### SUMMARY OF THE INVENTION

The present invention is directed towards methods of preventing or inhibiting the spread of viral infections by administering to a patient in need thereof a therapeutically effective amount of at least one Lactobacillus organism or by-product thereof and a pharmaceutically acceptable carrier. Vaginal administration of at least one Lactobacillus or by-product thereof in a pharmaceutically or food acceptable carrier, such as milk or portions thereof, including yogurt, provides a safe and effective means for treating, inhibiting the spread of, or reducing the occurrence of infections caused by viruses such as HIV, for example.

Another aspect of the present invention is directed to a method for preventing viral infection in mammals by coating a biosurface or biomaterial, such as an intra-uterine device or diaphragm, for insertion into a mammal with a anti-viral inhibitory amount of at least one Lactobacillus or by-product thereof.

In the practice of the methods of the present invention, the Lactobacillus may be administered as viable whole cells. The Lactobacillus species may be aerobically grown or microaerophillically grown and selected from L. rhamnosus, L. acidophilus, L. crispatus, L. fermentum, L. plantarum, L. casei, L. paracasei, L. jensenii, L. gasseri, L. salivarius, L. cellobiosis, L. brevis, L. delbrueckii, L. iners, L. rogosae and L. bifidum.

### DETAILED DESCRIPTION OF THE INVENTION

The present invention is directed to methods and compositions for treating, inhibiting or reducing the spread of viral infections in mammals caused by viruses including, but not limited to HIV, VSV and adenovirus by administration of one or more probiotic Lactobacillus whole cells or by-products thereof together with a pharmaceutically or food acceptable carrier. As defined by the present invention, a “probiotic” compound is a mono or mixed culture of microorganisms which when instilled in mammal, for example a human, affect the host beneficially. A preferred probiotic compound is Lactobacillus. As defined by the present invention a “by-product” of a Lactobacillus is a metabolic substance, not hydrogen peroxide, present in the culture fluid or supernatant obtained from lactobacilli grown in accordance with the present invention. A “by-product” of Lactobacillus is also understood in accordance with the present invention to include a biosurfactant. A biosurfactant is a compound released by lactobacilli, with a distinct tendency to accumulate at interfaces, most notably the liquid-air interface. Biosurfactant production can be measured conventionally by axisymmetric drop shape analysis by profile (ADSA-P).

Various physiological functions of biosurfactants have also been described. Biosurfactants can, inter alia, enable microorganisms to grow on water-immiscible compounds by lowering the surface tension at the phase boundary; biosurfactants can cause emulsification, and can stimulate adhesion of microbial cells to organic substrates. Biosurfactants have advantages over synthetic surfactants and it is those advantages that make biosurfactants prime candidates for industrial and biomedical applications. Biosurfactants are biodegradable and those from lactobacilli are non-toxic to humans. Biosurfactants have been discovered and disclosed previously (Reid et al. U.S. Pat. No. 6,051,552) ;however anti-viral activity of biosurfactants has not been previously considered.

Lactobacilli which can be vaginally instilled using the method described in the present invention can be administered as viable whole cells. The Lactobacillus can be aerobically or microaerophillically grown and selected from L. rhamnosus, L. acidophilus, L. crispatus, L. fermentum, L. plantarum, L. casei, L. paracasei, L. jensenii, L. gasseri, L. cellobiosis, L. brevis, L. salivarius, L. delbrueckii, L. rogosae, L. iners and L. bifidum. In a preferred embodiment, the Lactobacillus species are L. rhamnosus GR-1 (ATCC 55826), L. fermentum RC-14 (ATCC 55845) and L. fermentum B-54 (ATCC 55884), or L. reuteri RC-14.

In accordance with the present invention, vaginally administered Lactobacillus species or by-products thereof can colonize the human urogenital tract thereby competitively inhibiting and otherwise eradicating viruses extant in such environments and/or prevent the spread of such viruses, including but not limited to HIV, VSV and adenovirus. The vaginally administered Lactobacillus species can also stimulate the indigenous normal flora of the urogenital tract thereby preventing, treating and/or reducing the occurrence of infections caused by viral pathogens. The viral pathogens inhibited and otherwise depleted by the Lactobacillus and/or Lactobacillus by-products of the present invention include, but are not limited to, HIV, VSV, adenovirus type 5, varicella virus, herpes viruses, papilloma viruses and other viruses affecting the urogenital tract.

In a preferred aspect, the Lactobacillus is aerobically, microaerophilically or anaerobically grown and may be selected from the group consisting of Lactobacillus casei, L. acidophilus, L. plantarum, L. fermentum, L. brevis, L. jensenii, L. crispatus, L. rhamnosus, L. reuteri, L. paracasei, L. gasseri, L. cellobiosis, L. delbrueckii, L. helveticus, L. salivarius, L. collinoides, L. buchneri, L. rogosae, L. iners and L. bifidium.

The Lactobacillus may be microaerophilically or anaerobically grown. Any growth medium typically used to culture bacteria can be utilized. However, it is preferred that the cultures are grown in MRS broth. As they are growing in the growth medium, the lactobacilli are producing the by-products, such as biosurfactants.

The lactobacilli contemplated by the present invention are selected from the group consisting of Lactobacillus rhamnosus (GR-1 (ATCC 55826), L. rhamnosus GR-2 (ATCC 55915), L. rhamnosus GR-3 (ATCC 55917), L. rhamnosus GR-4 (ATCC 55916), L. rhamnosus RC-9, L. rhamnosus RC-17 (ATCC 55825), L. casei var alactosus RC-21, L. casei NRC 430, L. casei ATCC 7469, L. rhamnosus 81, L. rhamnosus 76, L. rhamnosus 36W, L. rhamnosus 36 g, L. casei RC-65, L. casei RC-15, L. casei 558, L. casei, RC-21, L. casei 55, L. casei 8, L. casei 43, L. plantarum RC-12 (ATCC 55895), L. acidophilus RC-25, L. plantarum RC-19, L. jensenii RC-11 (ATCC 55901), L. acidophilus ATCC 4357, L. acidophilus 2099 B, L. acidophilus 2155C, L. acidophilus T-13, L. acidophilus 1807B, L. acidophilus RC-16, L. acidophilus RC-26, L. acidophilus RC-10, L. acidophilus RC-24, L. acidophilus RC-13, L. fermentum RC-14, L. acidophilus RC-12, L. acidophilus RC-22, L. acidophilus 2099B, L. acidophilus 2155C, L. acidophilus T-13, L. plantarum ATCC 8014, L. plantarum UH 2153, L. plantarum 260, L. reuteri RC-14, L. plantarum RC-20, L. plantarum 75, L. plantarum RC-6, L. fermentum A-60, L. fermentum B-54 (ATCC 55920), L. iners CCP-1, L. cellobiosis RC-2, L. crispatus 1350B and L. crispatus 2142B.

The Lactobacillus useful in accordance with the practice of the present invention preferably attaches to human epithelial cells to a level of about 10 to 165 organisms per cell by hydrophobic, hydrophilic or other adhesion interactions.

Although this invention is not intended to be limited to any particular mode of application, vaginal administration of the compositions are preferred. One Lactobacillus organism or by-product thereof may be administered alone or in conjunction with a second, different Lactobacillus organism or by-product thereof. By “in conjunction with” is meant together, substantially simultaneously or sequentially. The compositions may be administered in the form of tablet, pill or capsule, for example. One preferred form of application involves the preparation of a freeze-dried capsule comprising the composition of the present invention. Another preferred form of application involves the preparation of a lyophilized capsule of the present invention. Still another preferred form of application involves the preparation of a heat dried capsule of the present invention. It has been found that a capsule comprising about 10⁹ lactobacilli is suitable. In accordance with the present invention a capsule may contain one single or two or more different species of lactobacilli and/or by-products thereof.

By “amount effective” as used herein is meant an amount of Lactobacillus or by-product thereof, high enough to significantly positively modify the condition to be treated but low enough to avoid serious side effects (at a reasonable benefit/risk ratio), within the scope of sound medical judgment. An effective amount of Lactobacillus or by-product thereof will vary with the particular goal to be achieved, the age and physical condition of the patient being treated, her race, the severity of the underlying disease, the duration of treatment, the nature of concurrent therapy and the specific Lactobacillus or by-product thereof employed. The effective amount of Lactobacillus or by-product thereof will thus be the minimum amount which will provide the desired anti-viral effect. For example, the presence of 1×10⁹ bacteria, as viable or non-viable whole cells, in 0.05 ml solution of phosphate buffered saline solution, or in 0.05 ml of suspension of microbial nutrients or prebiotics, or the dry weight equivalent of cell wall fragments, is effective when administered in quantities of from about 0.05 ml to about 20 ml. However, the presence of 0.05 ml to about 20 ml of Lactobacillus by-product solution in MRS broth produced from about 1×10⁹ bacteria is also effective.

A decided practical advantage is that the lactobacilli or by-products thereof may be administered in a convenient manner such as by the intravenous (where non-viable), suppository (vaginal or rectal) routes. Depending on the route of administration, the active ingredients which comprise the lactobacilli may be required to be coated in a material to protect said organisms from the action of enzymes, acids and other natural conditions which may inactivate said organisms. However, in the case of by-product administration, carriers rather than coatings may be required.

It is generally preferred that the by-products of lactobacilli are administered topically or coat or partially coat that portion of the biosurface or biomaterial that is inserted or placed into the desired locus of the urinary or vaginal epithelia. Any common topical formulation such as a solution, suspension, gel, cream, ointment, or salve and the like may be used. Preparation of such topical formulations is well described in the art of pharmaceutical formulations as exemplified, for example, in Remington's pharmaceutical Science, Ed. 17, Mack Publishing Company, Easton, Pa. (1988).

In addition to the Lactobacillus by-product described hereinabove, the compositions may additionally contain pharmaceutical vehicles, such as carriers and adjuvants described in the literature of pharmaceuticals, cosmetics and related fields.

A topical cream may be conventionally prepared as a semi-solid emulsion of oil in water or water in oil comprising the Lactobacillus by-products together with fatty alcohols, mineral oil or petrolatum and other typical pharmaceutical vehicles such as carriers, adjuvants, such as antioxidants, antiseptics and the like.

The by-products are present in the various pharmaceutical formulations described hereinabove in amounts effective to inhibit, reduce or prevent the spread of viruses such as HIV, for example. However, it is preferred that the formulation contains between 0.1 to 99 weight percent based on the total weight of the formulation for topical application. It is also preferred that the amount of the formulation of the present invention applied to a particular biosurface or biomaterial range from 0.001 μg to 100 μg/cm² relative to the area upon which the by-product is applied.

In another aspect of the present invention, a method for preventing viral infections in mammals is provided which involves coating a biologically compatible device with an effective amount of a Lactobacillus whole cell formulation or by-product thereof and inserting the device into the urogenital tract. The effective amount of Lactobacillus whole cell formulation or by-product coating is conventionally deposited on the outer surface of a biologically compatible device. The coating may also be conventionally applied to the inner surface of a device. The coating may be uniformly or non-uniformly deposited on the surface of a biologically compatible device. The biologically compatible device may be composed of polymers such as fluorinated ethylene propylene, sulfonated polystyrene, polystyrene, polyethyleneterephthalate silicone, polyurethane, polyvinylchloride silicone rubber, or glass, for example. The biodevice may be a catheter such as a urinary or peritoneal catheter, a diaphragm, a stent, an IUD or a diaper, an intravenous line, a peritoneal dialysis tube, an endotracheal tube, or an intravaginal, intrauterine, or intraurethral or intraureteral device, for example.

What has now been discovered, however, is that by-products produced by Lactobacillus species, in vitro, inhibited and eradicated viruses, such as VSV and adenovirus type 5 in several minutes.

In order to administer lactobacilli whole cells they should be coated by, or administered with, a material to prevent inactivation. For example, lactobacilli may be co-administered with enzyme inhibitors or in liposomes. Enzyme inhibitors include pancreatic trypsin inhibitor, diisopropylfluorophosphate (DFP) and trasylol. Liposomes include water-in-oil-in-water P40 emulsions as well as conventional and specifically designed liposomes which transport lactobacilli or their by-products to the urogenital surface. Dispersions can also be prepared, for example, in glycerol, liquid polyethylene glycols, and mixtures thereof, and in oils.

The pharmaceutical forms suitable for vaginal instillation also include sterile aqueous solutions (where water soluble) or dispersions and sterile powders for the extemporaneous preparation of sterile solutions or dispersions. The pharmaceutical forms for vaginal instillation must be stable under the conditions of manufacture and storage. The carrier can be a solvent or dispersion medium containing, for example, water, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride.

The lactobacilli and/or by-products thereof may conveniently be formulated into capsules or suppositories and may also contain the following: a binder such as gum tragacanth, acacia, corn starch or gelatin; excipients such as dicalcium phosphate; a disintegrating agent such as corn starch, potato starch, alginic acid, and the like; a lubricant such as magnesium stearate. When the dosage unit form is a capsule, it may contain, in addition to materials of the above type, a liquid carrier. Various other materials may be present as coatings or to otherwise modify the physical form of the dosage unit. For instance, capsules or lactobacilli in suspension may be coated with shellac, sugar or both.

The Lactobacillus is compounded for convenient and effective administration in effective amounts with a suitable pharmaceutically acceptable carrier in dosage unit form as hereinbefore disclosed. A unit dosage form can, for example, contain the principal active compound in an amount approximating I09 viable or non-viable.

The pharmaceutically acceptable carrier may be in the form of milk or portions thereof including yogurt. Skim milk, skim milk powder, non-milk or non-lactose containing products may also be employed. The skim milk powder is conventionally suspended in phosphate buffered saline (PBS), autoclaved or filtered to eradicate proteinaceous and living contaminants, then freeze dried heat dried, vacuum dried, or lyophilized. The carrier should be prepared to maximize the acidic effect of the lactobacilli or by-product(s).

Some other examples of substances which can serve as pharmaceutical carriers are sugars, such as lactose, glucose and sucrose; starches such as corn starch and potato starch; lactic acid, bacteriocin; cellulose and its derivatives such as sodium carboxymethycellulose, ethylcellulose and cellulose acetates; powdered tragancanth; malt; gelatin; talc; stearic acids; magnesium stearate; calcium sulfate; calcium carbonate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; cranberry extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tabletting agents, stabilizers, anti-oxidants and preservatives, can also be present.

The lactobacilli and/or by-products thereof of the present invention are administered in pharmaceutical compositions. The lactobacilli and/or by-products thereof of this invention are preferentially administered topically, e.g. to the urinary epithelia and vaginal epithelia alone or prior to insertion or placement of a biodevice such as a diaper, tampon, urinary catheter, intrauterine device, intravenous tube, dialysis tube, stent or diaphragm, for example.

Accordingly, in a preferred form of inhibiting or preventing Acquired Immune Deficiency Syndrome (AIDS), the patient is intravaginally administered a therapeutically effective amount of at least one Lactobacillus or by-product thereof and a pharmaceutically acceptable carrier in accordance with the present invention. Preferably, the Lactobacillus is selected from the group comprising L. rhamnosus, L. casei ss alactosus, L. fermentum and L. brevis. Most preferably, the Lactobacillus is either L. rhamnosus GR-1, L. fermentum B-54, L. fermentum RC-14, or L. reuteri RC-14.

Another aspect of the present invention is directed to the use of effective amounts of lactobacilli or by-products thereof to treat viral infections associated with the placement or insertion of biomaterials in contact with the urogenital tract, peritoneal space or blood stream, for example. The Lactobacillus or by-product thereof is applied topically in a virus-inhibiting amount to the patient. The specific anti-viral effective amount of Lactobacillus or by-product thereof is conventionally determined by the skilled artisan. Alternatively, the Lactobacillus or by-product thereof is applied onto or into the biomaterial in effective amounts prior to insertion into the infected area. Upon insertion thereof, the Lactobacillus or by-product thereof forms a coating on the infected area, thereby inhibiting further infection by the virus.

In still another aspect of the present invention biocompatible materials such as catheters, diapers, tampons, intrauterine devices, diaphragms, and stents or surfaces are coated with an effective amount of the by-product produced by lactobacilli to prevent the spread of viruses upon insertion into a mammal.

The amount of lactobacilli by-product utilized may vary depending upon various factors, including but not limited to the specific utility, whether the by-product is being applied to a biosurface or onto or into a biomaterial, and the like. The efficacious amounts used for the various utilities are conventionally determined by the skilled artisan. As indicated hereinabove, the by-products of the present invention are applied in effective amounts. Preferably, these amounts range from about 1 μg/ml to about 50 mg/ml, and more preferably from about 1 μg/ml to about 30 mg/ml.

In accordance with the present invention, metabolic by-products from Lactobacillus strains, including strains such as Lactobacillus rhamnosus GR-1 which does not produce hydrogen peroxide, have an inhibitory effect on human cells which reduces the risk of shedding of viruses upon sexual contact. Furthermore, the Lactobacillus whole cells and by-products thereof of the present invention also kill DNA and RNA viruses within seconds of exposure. Accordingly, if such DNA and/or RNAviruses are shed, or if they entered the vagina from sexual intercourse, such viruses are rapidly killed before infecting the non-infected host. In a preferred embodiment, the methods of the present invention are conducted in an environment created by the lactobacilli or by-products resulting in a microenvironment pH less than about 4.5.

In accordance with the present invention, the lactobacilli also inhibited yeast growth, thereby indicating the presence within the lactobacilli of substances which could control the ability of yeast within the urogenital tract to become the dominant and infecting organism.

In order to further illustrate the present invention, the experiments described in the following example was carried out. It should be understood that the invention is not limited to the specific example or the details described therein.

### EXAMPLE

Twenty nine healthy, Caucasian, premenopausal non-pregnant volunteers, with no symptoms or signs of urogenital infection, not receiving antibiotics, not using spermicides or immunosuppressive agents entered the study, which was approved by the Review Board for Health Sciences Research Involving Human Subjects at the University of Western Ontario. Each subject signed an informed consent. No changes to contraceptive practices were advocated and no subject became pregnant during the study. One gelatin capsule containing 10⁹ freeze dried Lactobacillus GR-1 and RC-14 was administered to 15 patients following menses. A second group of 14 women inserted one capsule containing 10⁹ freeze dried than Lactobacillus GG. Vaginal swabs were collected at day 0 (just after menses and before lactobacilli instillation), and 3, 7, 14 and 21 days following insertion of capsules and held at 4° C. until plating (within 8 hours). Saline dilutions of swab contents were plated onto MRS agar and MRS agar containing 8 μg/ml fusidic acid to aid in selection of RC-14 and GG/GR-1, respectively. The plates were incubated anaerobically for 48 hours at 37° C. Fifteen randomly selected Lactobacillus colonies from agar plates (containing 30-300 colonies) from each sample day were cultured in MRS broth overnight at 37° C. Lactobacillus genomic DNA was isolated and analyzed via RAPD-PCR as described elsewhere. RAPD fingerprints were compared to those of the probiotic strains for identification.

An agar overlay method was used to determine whether GR-1 and RC-14 inhibited Candida albicans growth, and GG was used as a negative control. In addition, survival of adenovirus 5 (Ad5)(non-enveloped double stranded DNA virus) and vesicular stomatitis virus (VSV)(an enveloped negative stranded RNA virus) was assayed as follows: the lactobacilli were grown for up to 24 hrs. in MRS broth, then 0.5 ml of their supernatant was added to 0.5 ml virus and incubated at 37° C. for 10 mins after which 0.5 ml was transferred to tissue culture plates for 1 hr. The culture fluid was aspirated and fresh media added for 20 hrs. (VSV) or 48 hrs. (Ad5). A plaque assay was then performed to measure viral infectivity. Controls included sterile MRS broth.

Results: There were no adverse events reported. Therapy with two strains (GR-1 along with RC-14) was significantly better in terms of recovery of one or both of the organisms versus therapy with only GG strain at day 14 (p=0.009). Six subjects were still colonized with GR-1 and/or RC-14 compared to only 2/14 with GG at day 21. L. fermentum RC-14 was only present in one women on day 21 and although it did not persist as long as the L. rhamnosus strains, it colonized for up to three days in 10/15 women. This illustrates, as we have previously shown (Gardiner, G., C. Heinemann, D. Beuerman, A. W. Bruce, and G. Reid. 2002. Persistence of Lactobacillus fermentum RC-14 and L. rhamnosus GR-1, but not L. rhamnosus GG in the human vagina as demonstrated by randomly amplified polymorphic DNA (RAPD). Clin. Diag. Lab. Immunol. 9: 92-96.) that lactobacilli strains, especially GR-1 and RC-14, can colonize the vagina. This is important because it thereby allows them to produce by-products, such as those described here with anti-viral effects.

Both GR-1 and RC-14 inhibited Candida albicans growth (inhibition zones of 2-4 mm). Culture fluid from GR-1 and RC-14 killed VSV and Ad5 within 10 minutes. Virus counts of 10⁹ VSV fell to 10⁴ with 12 hour pH 4.5 GR-1 supernatant and fell to zero with 24 hour pH 4 supernatants of both GR-1 and RC-14. Similar results were found for Ad5. MRS broth had no effect on viral viability.

Comments: The persistence of L. rhamnosus GR-1 on day 14 (11/15 subjects) compared L. rhamnosus GG (3/15), emphasized that not all probiotic strains, even of the same species, necessarily act optimally at the same host site. The anti-yeast effect corresponds to our experience of no cases of yeast vaginitis in more than 50 women using weekly vaginal lactobacilli therapy for up to one year, compared to an expected 200 episodes in these highly susceptible patients. The potent anti-viral activity seen here with Lactobacillus GR-1 and RC-14 fully supports the reduced risk of women acquiring sexually transmitted diseases including HIV, when colonized by lactobacilli. Given that women have an abnormal vaginal flora at many time points during their menstrual cycle, the ability to restore a lactobacilli-dominated flora using self-care products such as selected probiotics, represents a major breakthrough in women's health.

The observed effect was noted particularly when the pH of the environment was acidic, under pH 4.5, a value that is optimal for lactobacilli growth, survival and proliferation in the vagina. This is important in the anti-viral effects because bacterial vaginosis (the proliferation of organisms such as Gardnerella) and entry of sperm upon sexual intercourse, have alkaline properties which aid the transmission of viruses such as HIV. Thus, the acidic microenvironment produced by lactobacilli in conjunction with its anti-viral by-products, are vital in the effects described here.

Finally, the present invention is important for women wishing to take more control of their risk of viral infections, by self-administration of lactobacilli or their metabolic by-products. This is especially true in certain cultures where women have little access to condoms and pharmaceutical medications which might otherwise protect against such infections, or women who use spermicides which kill indigenous protective lactobacilli. Furthermore, the use of strains such as L. rhamnosus which survives exposure to spermicides, increases the ability of the therapy to reduce the risk of viral infection.

While not wishing to be bound by a particular mechanism wherein the inhibition of viral pathogenesis is achieved, it is believed that the results provided by the methods described herein are enhanced by pH effects. Such pH effects are observed with or without peptides or other molecules which kill the organisms, proteins, peptides, bacteriocins or other substances which inhibit viral binding to cells, alterations to host cells which change their receptivity to viral entry. These methods are different from immune modulation or hydrogen peroxide effects described previously.

What is claimed:

1. A method for inhibiting the spread of viral infection in a vaginal tract of a subject, comprising administering a therapeutically effective amount of a pharmaceutical composition, wherein said composition comprises at least one Lactobacillus and a carrier.

2. The method of claim 1, wherein said Lactobacillus is a whole cell or metabolic by-product thereof.

3. The method of claim 1, wherein said Lactobacillus is selected from the group consisting of L. rhamnosus GR-1 (ATCC 55826), L. fermentum RC-14 (ATCC 55845) and L. fermentum B-54 (ATCC 55884).

4. The method of claim 2, wherein said by-product is biosurfactant.

5. The method of claim 1, wherein said infection is caused by viruses selected from the group consisting of HIV, VSV and adenovirus, varicella virus, herpes viruses and papilloma viruses.

6. The method of claim 1, wherein said composition is administered vaginally.

7. A method for preventing the spread of viral infection in a vaginal tract of a subject, comprising coating a biologically compatible device with an effective amount of a pharmaceutical composition and inserting the device into the urogenital tract, wherein said composition comprises at least one Lactobacillus and a carrier.

8. The method of claim 7, wherein said Lactobacillus is a whole cell or metabolic by-product thereof.

9. The method of claim 7, wherein said Lactobacillus is selected from the group consisting of L. rhamnosus GR-1 (ATCC 55826), L. fermentum RC-14 (ATCC 55845) and L. fermentum B-54 (ATCC 55884).

10. The method of claim 8, wherein said by-product is biosurfactant.

11. The method of claim 7, wherein said infection is caused by viruses selected from the group consisting of HIV, VSV and adenovirus, varicella virus, herpes viruses and papilloma viruses.

12. The method of claim 7, wherein said biologically compatible device is selected from the group consisting of a urinary or peritoneal catheter, a diaphragm, a stent, an IUD or a diaper, an intravenous line, a peritoneal dialysis tube, an endotracheal tube, or an intravaginal, intrauterine, intraurethral or intraureteral device.

13. A method for treating a viral infection in a vaginal tract of a subject, comprising administering a therapeutically effective amount of a pharmaceutical composition, wherein said composition comprises at least one Lactobacillus and a carrier.

14. The method of claim 13, wherein said Lactobacillus is a whole cell or metabolic by-product thereof.

15. The method of claim 13, wherein said Lactobacillus is selected from the group consisting of L. rhamnosus GR-1 (ATCC 55826), L. fermentum RC-14 (ATCC 55845) and L. fermentum B-54 (ATCC 55884).

16. The method of claim 14, wherein said by-product is biosurfactant.

17. The method of claim 13, wherein said infection is caused by viruses selected from the group consisting of HIV, VSV and adenovirus, varicella virus, herpes viruses and papilloma viruses.

18. The method of claim 13, wherein said composition is administered vaginally.

19. A topical cream comprising at least one Lactobacillus by-product in a semisolid emulsion and a pharmaceutical vehicle or adjuvant, wherein said emulsion is selected from oil in water or water in oil and wherein said pharmaceutical vehicle or adjuvant is selected from the group consisting of fatty alcohol, mineral oil or petrolatum, antioxidants or antiseptics.

20. The method of claim 19, wherein said Lactobacillus is selected from the group consisting of L. rhamnosus GR-1 (ATCC 55826), L. fermentum RC-14 (ATCC 55845) and L. fermentum B-54 (ATCC 55884).

21. The method of claim 19, wherein said by-product is biosurfactant.

22. A capsule or suppository comprising a pharmaceutical composition and further comprising at least one second composition, wherein said pharmaceutical composition comprises at least one Lactobacillus and a carrier, and wherein said second composition is selected from the group consisting of a binder, an excipient, a disintegrating agent, a lubricant, a liquid carrier or a combination thereof.

23. The method of claim 22, wherein said Lactobacillus is a whole cell or metabolic by-product thereof.

24. The method of claim 22, wherein said Lactobacillus is selected from the group consisting of L. rhamnosus GR-1 (ATCC 55826), L. fermentum RC-14 (ATCC 55845) and L. fermentum B-54 (ATCC 55884).

25. The method of claim 23, wherein said by-product is biosurfactant.

26. The method of claim 22, wherein said infection is caused by viruses selected from the group consisting of HIV, VSV and adenovirus, varicella virus, herpes viruses and papilloma viruses.

27. The method of claim 13, wherein said composition is administered orally or in form of suppository.
